(19) United States
(12) Patent Application Publication
(10) Pub. No.: US 2008/0027358 A1
(43) Pub. Date: Jan. 31, 2008

(54) Morphometry of a Bodily Hollow System

(75) Inventors: Hans Gregersen, Hornslet (DK); Flemming Gravesen, Aalborg (DK)

Correspondence Address: KNOBBE MARTENS OLSON & BEAR LLP, 2040 MAIN STREET, FOURTEENTH FLOOR, IRVINE, CA, 92614, US

(73) Assignee: DITENS A/S, Aalborg (DK)

(21) Appl. No.: 11/630,527

(22) PCT Filed: Jun. 30, 2005
(86) PCT No.: PCT/DK05/00449

§ 371 Date: Feb. 7, 2007

(30) Foreign Application Priority Data
Jun. 30, 2004 (DK) PA 2004 01034
Aug. 31, 2004 (DK) PA 2004 01316
Aug. 31, 2004 (DK) PA 2004 01318

(51) Int. Cl. A61B 5/103; A61B 5/04; A61B 5/05

(52) U.S. Cl. 600/593; 600/407; 604/509

(57) ABSTRACT

The present invention relates to the determining of morphometric properties of bodily hollow systems at the natural state or during distension of the organ using a balloon or bag. A method of obtaining morphometric measures of a hollow internal organ is disclosed. The method comprising the steps of introducing from an exteriorly accessible opening of a bodily hollow system a catheter into the hollow system, the catheter being provided with one or more inflatable balloons situated between a proximal end and a distal end of the catheter, subsequently inflating at least one of the balloons in the hollow internal organ at least until the balloon abuts an inner wall of the hollow system and determining at least one morphometric parameter at a level of inflation. Moreover, the invention relates to an apparatus for measurement of morphometric data of a bodily hollow system.

### FIELD OF THE INVENTION

The present invention relates to the determining of morphometric properties of bodily hollow systems at the natural state or during distension of the organ using a balloon or bag.

### BACKGROUND OF THE INVENTION

The function of visceral organs like the gastrointestinal tract, the urinary tract and the blood vessels is to a large degree mechanical which depends on the morphometric properties of the organ. The following introduction refers mainly to the gastrointestinal tract but the invention has similar applications in other hollow organs.

In the gastrointestinal tract, contents received from the stomach are propelled further down the intestine and mixed with secreted fluids to digest and absorb the food constituents. The biomechanical properties of the small intestine in vivo are largely unknown, despite the fact that the distensibility is important for normal function, and altered mechanical properties are associated with gastrointestinal (GI) diseases. Data in the literature pertaining to the mechanical aspects of GI function are concerned with the contraction patterns, the length-tension relationship in circular and longitudinal tissue strips in vitro, flow patterns, the compliance and the tension-strain relationship. The methods traditionally used for clinical or basic investigations of the small intestine are endoscopy, manometry and radiographic examinations. Although these methods provide important data on the motor function, little attention has been paid to biomechanical parameters such as wall tension and strain and the relation between biomechanical properties and sensation. During the past two decades, impedance planimetry was used in gastroenterology to determine wall tension and strain in animal experiments and human studies. Impedance planimetry provides a measure of balloon cross-sectional area and is therefore a better basis than volume measurements for determination of mechanical parameters such as tension and strain in cylindrical organs.

GI symptoms are often associated with disturbances in motility and sensory function in the GI tract. Several studies attempted to investigate these properties by means of balloon distension. Unfortunately, the primary mechanism for symptoms elicited by GI distension remains unclear. It is well known that distension of the gastrointestinal tract elicits reflex-mediated inhibition and stimulation of motility via intrinsic or extrinsic neural circuits and induces visceral perception such as pain. Previous studies demonstrated that mechanoreceptors located in the intestinal wall play an important role in the stimulus-response function. It is, however, a common mistake to believe that mechanoreceptors are sensitive to variation in pressure or volume. A large variation in the peristaltic reflex and perception have been found in various studies and species suggesting that pressure is not the direct stimulus. Instead, the receptors are stimulated by mechanical forces and deformations acting in the intestinal wall due to changes in the transmural pressure. Thus, the mechanical distension stimulus and the biomechanical tissue properties must be taken into account in studies of the sensory-motor function in the intestine. For the same reason the morphometric properties must be known.

Abdominal discomfort is among the most common symptoms responsible for patients consulting the health care system. More knowledge on the relation between sensation and the morphometric and biomechanical properties of the gastrointestinal tract is obviously needed to increase our understanding and to improve treatment of these patients.

Abdominal discomfort and pain are the products of a multi-dimensional perception with highly individual cognitive, emotional and social aspects. Therefore, standardized experimental studies in healthy volunteers and selected patient groups are needed. In these studies the exact nature of the stimulus (bag distension, warmth, cold, electricity, chemical substances or a multi-modal combination) has to be controlled. The most widely used method for visceral stimulation is mechanical distension of hollow viscera with balloons/bags. Distension of the gut activates mechano-sensitive afferents in the wall structure, i.e. mucosa, muscle layer and serosa. Several studies indicate that the mechano-receptors are not directly sensitive to volume or pressure. Instead the degree of tissue deformation (strain) and mechanical forces (tension and stress) seem to be the direct receptor stimulus. An analytical tool for assessment of these biomechanical wall parameters during bag distension is needed.

It is well known that the passive elastic behaviour of biological tissues is exponential. The exponential behaviour protects the organs including the intestine against overdistension and damage at high luminal pressure loads and allows the intestine to distend easily to facilitate flow in the physiological pressure range. In arteries, it has been demonstrated that collagen bears circumferential loads at high stress levels. Since gastrointestinal tissue is rich in collagen, it is likely that collagen is a major determinant of the curve shape. The passive elastic behaviour (tension-strain relation) of duodenum in vivo is exponential and hence can play a role in protecting tissue against high stress. At high loads the mechanical behaviour is contributed mainly by the passive tension curve, whereas at low stress levels, that is in the physiological range, the active tension curve also affects the tissue behaviour. Thus, the distensibility in vivo depends not only on the passive properties but also on the physiological state of smooth muscle.

Mechanical properties have been studied in vitro in muscle tissue strips from various organs. The strips are mounted in a small organ bath between hooks so the strip can be elongated in a controlled way and the resultant force measured. This has made possible studies of isometric and isotonic muscle length-tension diagrams in vitro. Usually the tissue has been studied when influenced by drugs such as muscle relaxants and muscle stimulants, in order to study active and passive tissue properties. The passive curve is normally described as exponential whereas the active curve is bell-shaped, i.e. with a maximum. The maximum active tension is presumably reached at a level of optimum overlap between the sliding filaments in the intestinal muscle cells. In vivo no such method exists. Manometry is used to record the contraction patterns but gives no information about the passive mechanical properties and only indirect data on the force of contraction. Balloon distension techniques with recording of balloon pressure and balloon dimensions such as volume and cross-sectional area can provide a mechanical stimulus to the wall but in the way these techniques have been used, data on the smooth muscle force have been sparse and control of passive conditions have been insufficient.

During the past centuries several methods for assessment of the biomechanical wall properties of hollow viscera have been established. The first studies were based on ex vivo muscle strips measurements assessing the strain, tension and stiffness of the resected wall material. Biomechanical data of the in vivo gastrointestinal tract were traditionally based on pressure-volume measurements obtained during bag distensions (barostat method). The method of bag distension has been refined by the introduction of the impedance planimetric technique. This is now a well-established method, which allows the circumferential strain, tension (applying the Laplace's Law) and stiffness (slope of tension-strain relation) to be computed.

Each method has several advantages and limitations. Further development of the bag distension technique and analysis is needed for complete description of the morphometric and biomechanical wall properties. A new method based on simultaneous cross-sectional imaging (such a ultrasonography, computed tomography (CT) and magnetic resonance imaging (MRI)) and pressure measurement during bag distension gives the possibility for modelling of the three-dimensional geometry of the gastrointestinal tract. This includes the spatial distribution of the three-dimensional principal curvatures, radii, wall thickness, tension and stress. Since the mechano-sensitive receptors are probably responding to the entire three-dimensional (circumferential, longitudinal, radial and shear) deformation and mechanical forces of the wall structure this approach can be valuable.

### SUMMARY OF THE INVENTION

This invention comprises a method and apparatus to obtain important data on the distribution of morphometric parameters, thermal properties (tissue perfusion), and mechanical properties in the organ with or without previous or simultaneous stimulation, such as with balloon distension (in this script the term balloon also cover the use of a non-compliant large bag).

Two levels of inflation are defined where the morphometric parameters are obtained, the first level being filling the bag and thereby the organ but without significant stretch of the wall, and the second level where wall stretch occur. The levels can be distinguished by means of pressure, volume, cross-sectional area, tension or similar curves where for example the pressure at the first level will be steadily low, but rise steeply at the second level.

In a preferred embodiment the bag inflation is combined with imaging technology to obtain morphometric data such as wall thickness; layer thickness; inner and outer surface areas; areas of interfaces between layers, inner and outer circumferences; circumferences of interfaces between layers; wall volume; layer volume; wall cross-sectional area; layer cross-sectional area; luminal cross-sectional area; luminal diameter, or other measures.

In another embodiment, the balloon has electrodes on the outside for detection of tissue impedance/conductance or other sensors such as pH-sensors. This can, with or without simultaneous effect of drugs or other stimulations, provide data on wall layers, contractile activity, oedema, and ischemia during distension. The invention also covers algorithms for estimation of the cross-sectional areas, balloon shape and mechanical parameters, calibrations, data transmission between the catheter and the hardware, simulation, etc. The method can be combined with administration of drugs and chemicals locally or systemically. The invention also covers various distension protocols and the correlation of the above parameters with sensory data as reported on visual analogue (VAS) scales, electrical brain potentials and referred pain.

The invention can be used to follow the progress of a disease or pharmaceutical treatment. Of interest is diagnosis and progress and distribution of celiac disease, systemic sclerosis, obstruction, inflammation, crohns disease, other inflammatory diseases, fibrosis, diverticulosis, linitis plastica in the digestive tract and a number of other diseases associated with other organ systems.

In one embodiment the temperature of the balloon can be changed in a controlled way, either as a step or a ramp, and with or without changing the fluid volume in the balloon. Such data will provide important information about the tissue perfusion and the ability to change the balloon temperature back to body temperature. It is believed that a high perfusion is a means of protection of the tissue against chemicals and other stimulations. With knowledge about the contact surface between the tissue and the balloon it is possible to compute a heat or cold flux. The contact surface area can be estimated by imaging means or from volume or multiple cross-sectional area measurements. Such information may be important for tissue perfusion and hence for the evaluation of esophagitis, ulcers, inflammation, and pain mechanisms.

This invention comprises a further development of balloon distension methods by providing force-deformation diagrams such as tension-strain measures in vivo before and during administration of muscle relaxant or muscle stimulating drugs. Such data may be obtained by means of pressure recordings combined with impedance planimetry for measurement of the mid-balloon cross-sectional area or various intraluminal or external imaging technologies. Hereby, active and passive properties can be studied in vivo and can be related to other physiological responses such as to pain elicited by the mechanical stimulation. Development of balloon distension protocols is useful in order to correlate biomechanics, motor control and visceral non-pain and pain perception in the visceral organs, in particular in the gastrointestinal tract in vivo and in vitro. The distension can be used to derive isometric length-tension data in vivo with subsequent evaluation of the circumferential wall tension, strain and sensory intensity. This length-tension test provides data on the passive nature of the tissue, on the maximum force generated by the smooth muscle, and the strain corresponding to the maximum force. Furthermore, pressure-area loops will serve as an organ function test and force-velocity data can be obtained by analysing pressure-CSA or tension-strain data from individual contractions during balloon distension.

### DESCRIPTION OF THE DRAWINGS

FIG. 1 shows pressure-volume data obtained during distension of a rat stomach. Level 1 during the inflation continues into level 2 approximately at a pressure of 200 Pa.

FIG. 2 shows examples of ultrasonographic scanning of the esophagus during bag distension to various VAS levels. The morphometry in terms of wall thickness, wall layers, etc can clearly be distinguished.

FIG. 3 shows example of circumferential stretch ratios at various VAS levels at the esophageal mucosa, interface between muscle layer and submucosa and at the outside of the longitudinal muscle layer. Data are obtained by endoluminal ultrasonography.

FIG. 4 shows an example of distal stomach wall thickness as function of bag volume.

FIG. 5 is an illustration of a ramp distension curve in the human esophagus. The thick line at the end of the curve is the reverse point. The open symbols above the curve mark the phasic part ( - - - ) and the afterload pressure. The closed symbols under the curve mark the tonic part ( - - - ) of the distension curve and the preload pressure. The passive pressure is measured from the tonic part of the curve during administration of butylscopolamine. At the symbols pressure and CSA was measured and radius and tension was computed from these values.

FIG. 6 illustrates volume, radius, and pressure as function of time during the distensions without (left panels) and during (right panels) the administration of butylscopolamine.

FIG. 7 illustrates the active and passive tension-radius curves for a subject where the maximum active tension is reached before the moderate pain level (top). The bottom graph shows the averaged data from all volunteers. Mean and SEM values are shown.

FIG. 8 provides a representation of the change in muscle tension during distension-induced contractions as function of the radius immediately before the contraction (preload-afterload properties) of the esophageal muscles. The solid line is the polynomial fit and the dotted lines represent 95% confidence intervals.

FIG. 9 illustrates tracings obtained during a distension in a patient with systemic sclerosis. This patient only has slight hypomotility. The two upper tracings show the cross-sectional area and pressure during the whole bag filling phase. The two bottom curves show the radius and the pressure from only a part of what is shown in the upper curves. The arrows inserted in the radius curve show that the slope decrease when the load is increased.

FIG. 10 shows force-velocity curves and force-power curves represented as circumferential preload tension-radius shortening velocity (A) and preload tension-circumferential preload tension*CSA rate (B) in SS patients (blue lines) and controls (red lines). The presented scatter data were from whole SS patients (◯) and controls (□). The tension-velocity data were curve fitted by using Hill's equation and the power-tension data were fitted by using cubic polynomial function.

FIG. 11 shows examples of esophageal pressure-CSA and tension-radii loops.

FIG. 12 depicts a diagram of a thermal stimulation system using a peristaltic pump. The arrows show the flow of the water.

FIG. 13 shows an example of temperature experiments in the human esophagus where the temperature initially is 60 degrees Celsius and where it drops as function of time at volumes 10, 15 and 20 ml inside the bag.

FIG. 14 schematically illustrates a catheter into the esophagus of a person.

### DETAILED DESCRIPTION OF THE INVENTION

FIG. 14 schematically illustrates a catheter, the catheter being provided with an inflatable balloon (the catheter may be provided with more balloons), the balloon(s) being situated between a proximal and a distal end of the catheter. The catheter and thereby the balloon is inserted into the esophagus of a person. The catheter is inflated to a given level, so as to exert a pressure on the esophagus and thereby imposing a morphological change to the esophagus. This causes the muscles surrounding the esophagus to react by trying to drag the balloon and the probe away from the tract.

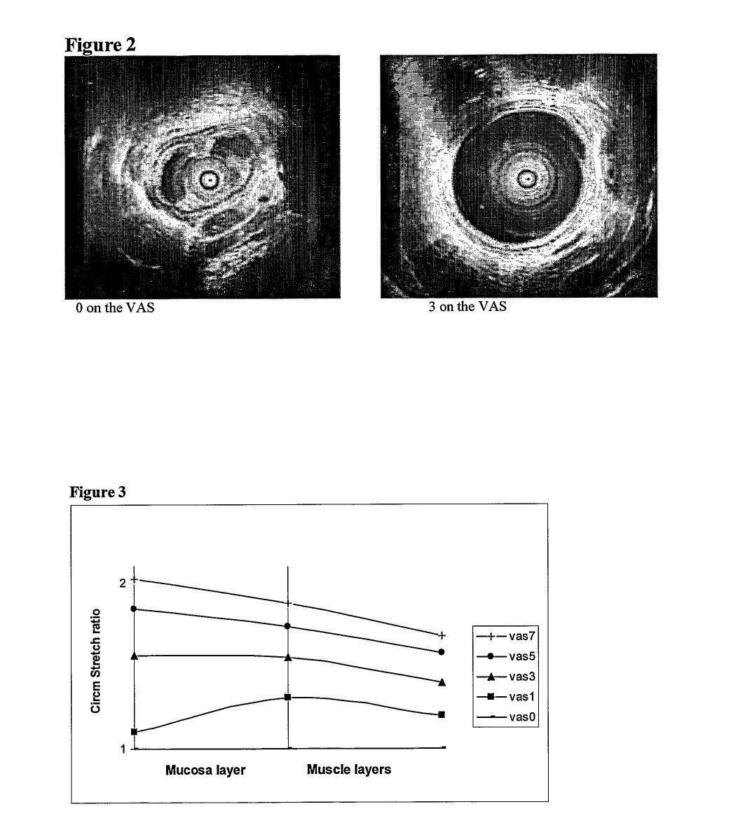

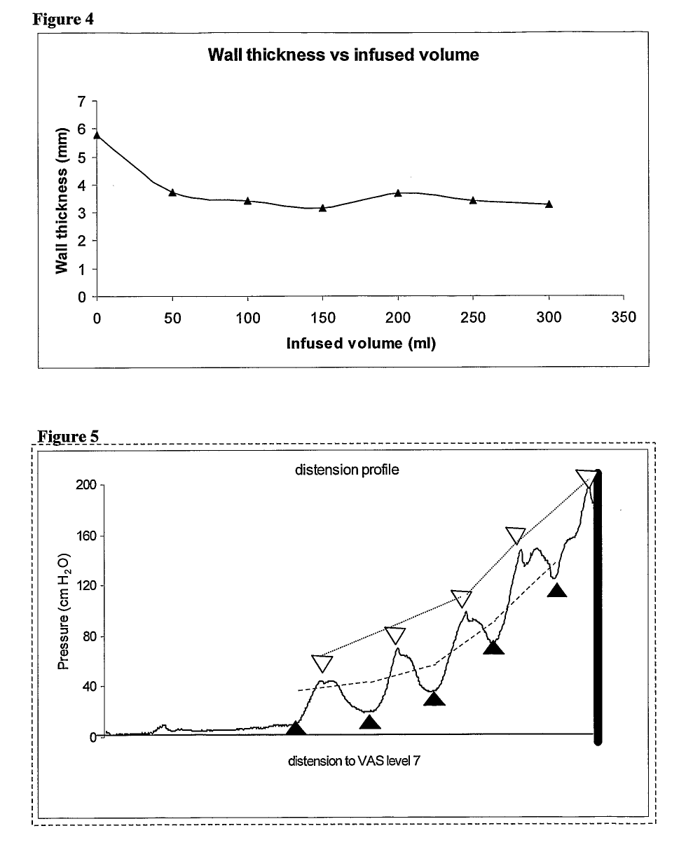

The method and apparatus serve to determine physical properties of hollow internal organs comprising means for determining morphometric parameters, thermal measures, and mechanical parameters. In a preferred embodiment morphometric properties will be obtained by analysis of medical images, often serial slices of 2D images, as illustrated in FIG. 2, with respect to measures such as wall thickness (FIG. 4); layer thickness; inner and outer surface areas; areas of interfaces between layers, inner and outer circumferences (FIG. 3); circumferences of interfaces between layers; wall volume; layer volume; wall cross-sectional area; layer cross-sectional area; luminal cross-sectional area; luminal diameter, or other measures. FIG. 2 shows ultrasound generated images that can be used as basis for a morphometric and mechanical analysis of the esophagus. From FIG. 2 the circumferences, layer and wall thicknesses can be measured. The measures will be obtained in the entire organ or part thereof using medical imaging technique such as magnetic resonance scanning, X-rays and fluoroscopy in one or more planes, CT scanning, ultrasound or other imaging means. The analysis comprises reconstruction of the organ using various algorithms, detection of surfaces and interfaces between layers and organs with secondary derivation of the parameters mentioned above and possibly reconstruction and colour coding for better visualization. The analysis can be taken into the time domain.

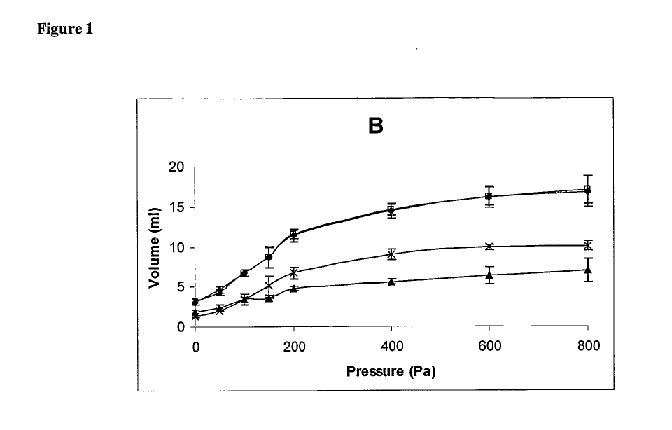

In a preferred embodiment, the system comprises a balloon attached to a catheter. The balloon can be inflated with a fluid, such as gas (e.g. air) or a liquid, or under the influence of relaxing or stimulating drugs or chemicals. The balloon distension can be combined with imaging technology for measurement of pressure, dimension or other parameters, or for infusion of liquid or air into the organ under study. Morphometric parameters such as volume, luminal cross-sectional area, diameter, circumferences, layer and wall thicknesses, and pressure are measured during inflation of a balloon, thereby inducing strains, tensions and stresses applied by the balloon or bag to the internal surface of the wall of the hollow system. The inflation of the balloon may be done until a given level, so that at least one morphometric parameter can be determined at a level, such as at a first and second level, or possible at various levels. The first level may be where the balloon in the hollow organ abuts an inner wall, and the second level may be where the inflation of the balloon is such that a pressure from the balloon is exerted on the inner wall of the hollow organ. Thus, two levels of inflation are defined where the morphometric parameters may be obtained, the first level being filling the bag and thereby the organ but without significant stretch of the wall, and the second level where wall stretch occur. The levels can be distinguished by means of pressure, volume, tension or similar curves where for example the pressure at the first level will be steadily low, but rise steeply at the second level. FIG. 1 is an illustration of how level 1 and level 2 can be separated. In the data illustrated in the figure a pressure of 200 Pa is a natural separation point between the filling phase (level 1) and the distension phase (level 2).

In one embodiment the length of the balloon or bag can be changed by way of a system where part of the balloon or bag is closed off by means of a string system, a smart device or similar. This allows for studies using different and controlled length of the balloon during the experiments.

An embodiment comprises determination of the balloon size and shape from cross-sectional area measurements from intraluminal or externally placed ultrasound or MR coils, including determination of angles between the cross-sectional areas measured by the probe. Tensions, stresses and strains or elastic stiffness in one or more directions and possibly in different layers can be evaluated from equilibrium analysis or by finite element analysis of the data obtained from the multiple geometry measurements such as multiple cross-sectional areas along the longitudinal axis of the catheter.

In one embodiment with a balloon placed in the lumen of an organ, electrodes are placed on the outside of the balloon for measurement of one or more wall impedances or conductances during distension or deflation of the balloon or bag. The wall impedance/conductance provide information about the organ function and can be correlated to the degree of balloon distension or derived mechanical parameters hereof at resting conditions or during natural movement such as swallows in the esophagus or during physical stimulation with infused volumes, electrical stimulation, chemical stimulation, systemic or local infusion of drugs or other artificial stimulation means to provide information about the organ properties. In some cases it can also be used to estimate the thickness of the wall and using pharmacological substances even layer thicknesses using parallel conductance theory. The electrodes may be placed in ways so information is gained about the properties in different directions.

Any kind of data signal and possibly also the energy (activation current) provided for measurements of various parameters such as cross-sectional area can be transmitted wireless using infrared light, Bluetooth, external electromagnetic radiofrequency source (RF) signals or other means from the probe to the hardware system or from an intermediate data acquisition box driven by a power supply or by batteries wireless to the hardware box. This will minimize electrical hazards.

Since the probes may have a unique distance between the electrodes and other parameters are known such as the conductivity of the fluid, then the probes can be precalibrated. This means that the equipment can identify a specific type of probe or the user can load the calibration from a file. Other signals such as VAS data can also be precalibrated. In one embodiment a chip or other solution is used to match the catheter to the data acquisition system. This will avoid the use of non-authorized catheters.

One embodiment allow mechanical parameters such as tensions, stresses and strains to be computed online and used in a feedback system with other measures such as VAS data, electrical brain signals, electronic registration of referred pain data, longitudinal force measurements, and luminal pressures to provide a feedback system for safety and running standardized protocols. Distension protocols is important in mechanical studies and the analysis of data combining unique protocols such as preconditioning protocols with the organ behaviour and the subject behaviour such as the change in VAS score from the first to the second mechanical distension. Other unique protocols are used to monitor organ and subject behaviour such as first preconditioning the tissue in a rapid fashion, then run mechanical tests and other tests such as electrical thermal or chemical stimulations in a prearranged way. These are merely examples of protocols.

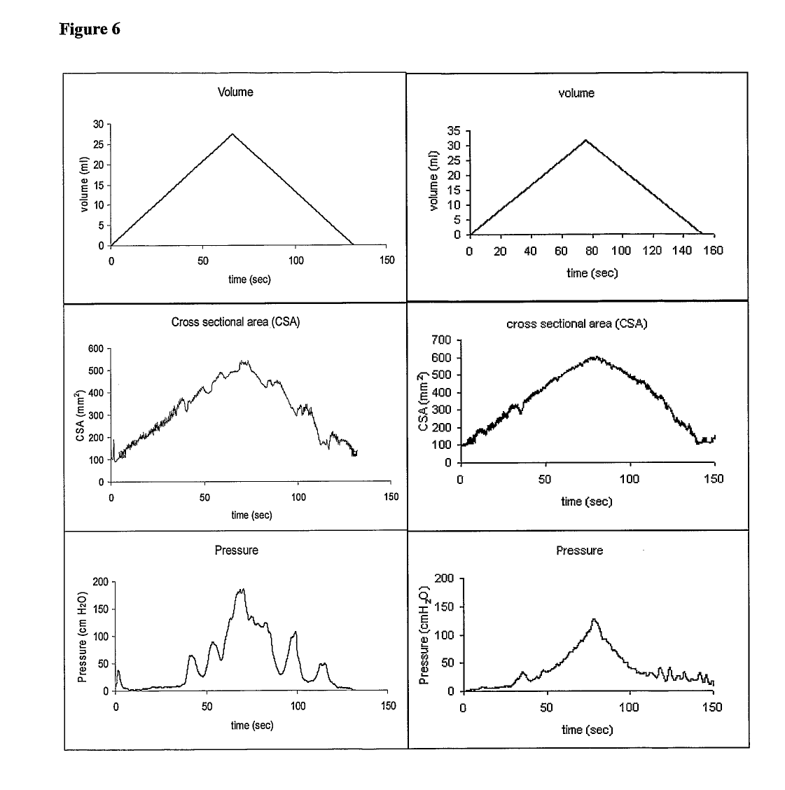

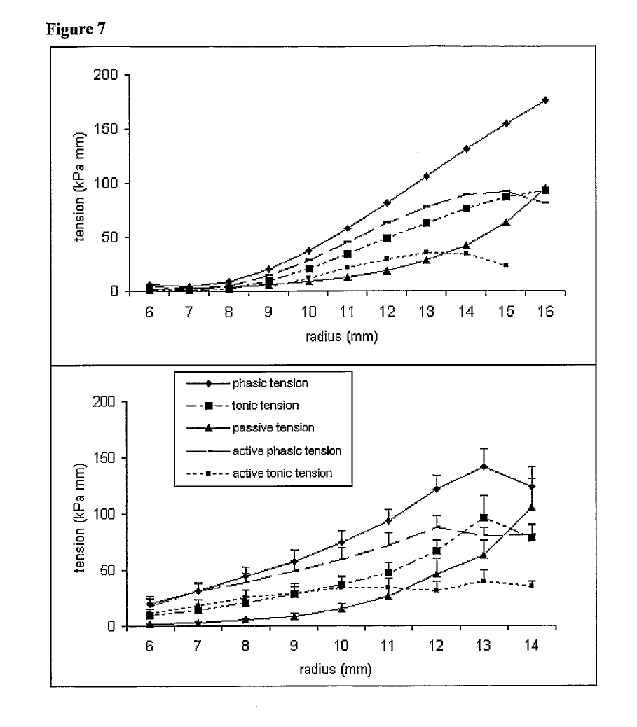

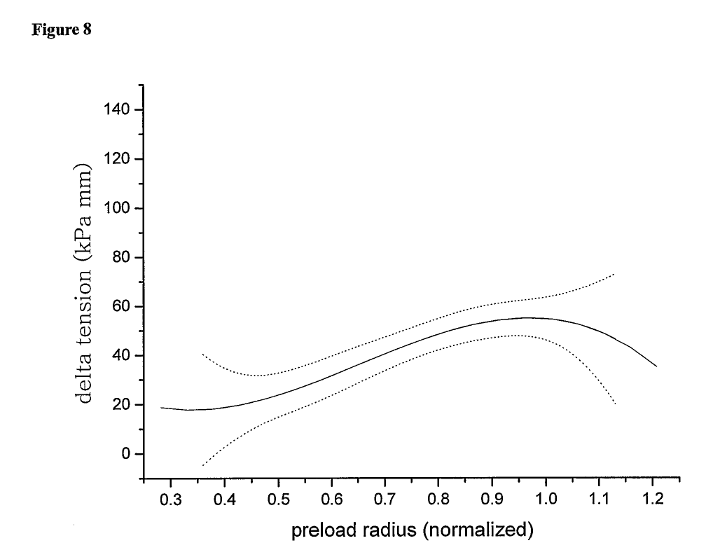

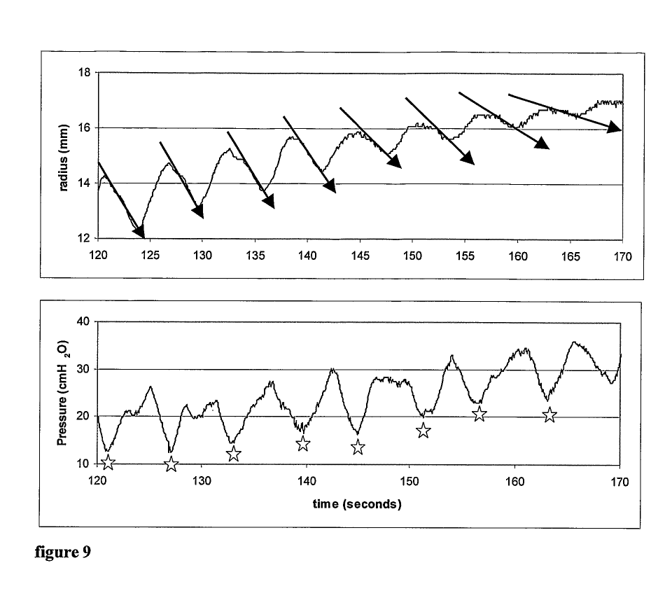

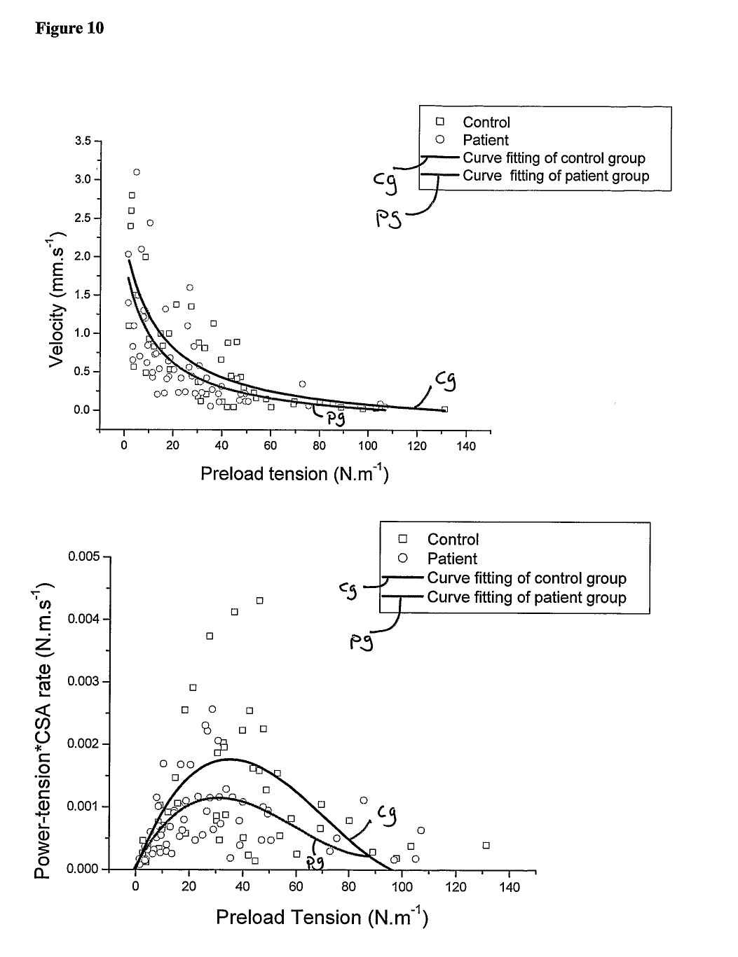

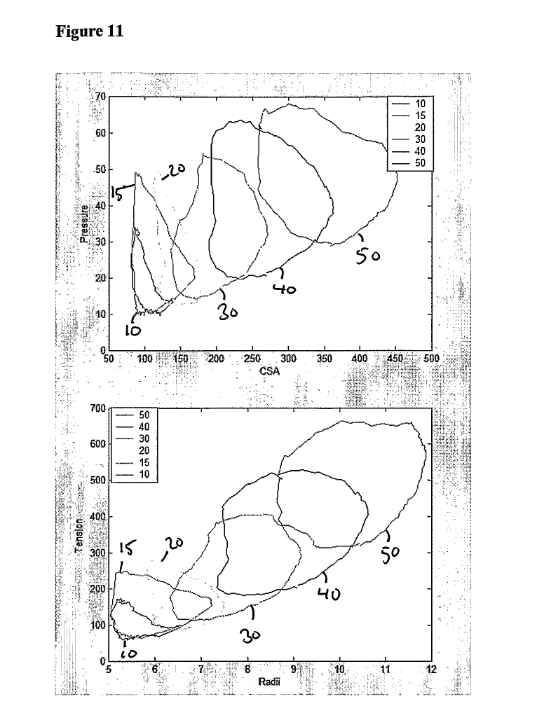

In one embodiment the morphometric data and other data such as pressures and forces are used for computation of advanced mechanical parameters such as active-passive tension-strain analysis (FIG. 7), active-passive stress-strain analysis, delta tension-preload radius (FIG. 8), tension-velocity plots (FIG. 10), power plots (FIG. 10), preload-afterload muscle analysis (FIG. 8) using algorithms in real time of offline with or without correlation to sensory data as obtained by VAS scales or other means. Other analysis relates to pressure-CSA loops and tension-radius loops during contractions (FIG. 11). Such data may be obtained by means of pressure recordings combined with impedance planimetry for measurement of the mid-balloon cross-sectional area or various intraluminal or external imaging technologies. FIGS. 3-11 show various mechanical analysis and plots.

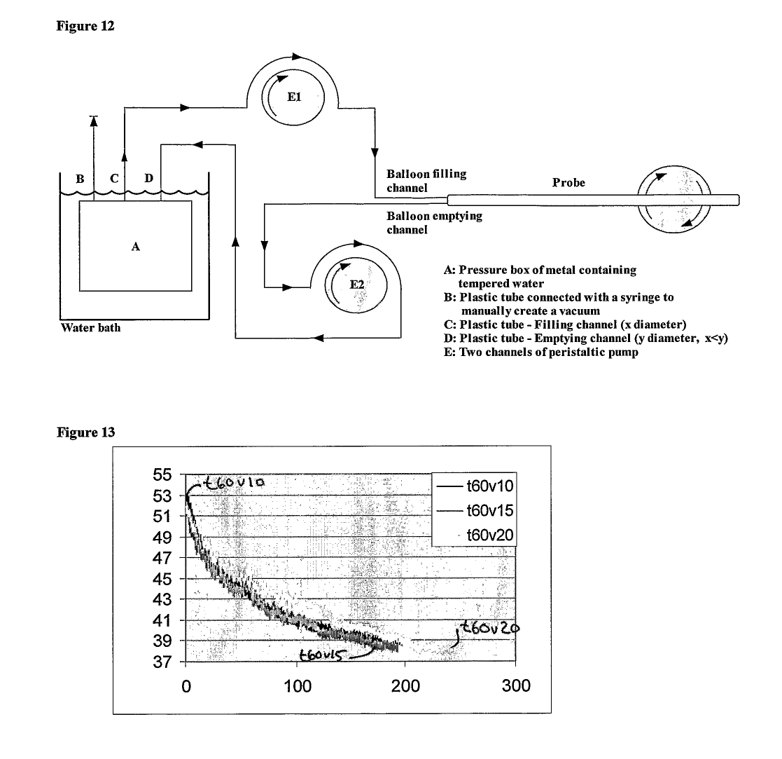

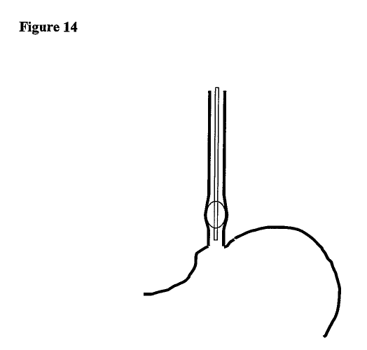

In one embodiment the temperature of the fluid in the balloon can be changed in a controlled way, preferably in a step fashion with or without changing the volume inside the balloon, and measured by temperature sensors placed inside or on the surface of the balloon or bag to analyze the temperature change and time to reach a baseline as indicative of the tissue perfusion (FIGS. 12-13). The response is indicative of tissue perfusion, a parameter that may be important in various diseases such as esophagitis. The temperature test can be done at various degrees of distension with analysis of temperature change parameters as function of morphometric and mechanical measures such as volume, cross-sectional area, diameter, length, pressure, force, tensions, stresses and strains. If the contact area between the tissue and balloon is known, the temperature (heat and cold) flux can be determined.

The methods in the above embodiments are used in combination with an apparatus. For the balloon embodiments an apparatus for measurement of multiple cross-sectional areas by imaging technology and other parameters inside bodily hollow systems and sensory parameters, the apparatus comprising a catheter being provided with an inflatable balloon situated between a proximal end and a distal end of the catheter, and the apparatus comprising means for passing an inflating fluid, preferably a liquid, from the proximal end to the balloon, and the apparatus furthermore comprising means for establishing a first and a second level of inflation of the balloon by measurement of cross-sectional areas, pressures or volume. The apparatus may in an embodiment comprise means for measurement of cross-sectional areas, pressures, layer and wall thicknesses, electrical impedance, conductance or pH on the outside of the balloon. The catheter may use different types of wiring and electrodes such as wires, printed flexible circuits or silk prints for inducing or detecting electrical parameters. The apparatus may use wireless transmission and/or energy transmission between the catheter and the hardware, or data processing unit or between an intermediate box, or intermediate unit, and the hardware and it may include means to match the catheter to the hardware. The apparatus may also include an automated or manual pump and heating/cooling system for inflation of the balloon and for providing temperature stimuli. The system may comprise all algorithms and analysis tools for providing feedback, patient security, calculations, sensory responses, etc.

Example of an Apparatus and Experiment with Imaging Technology

The following states an example of experiment and analysis of morphometric and mechanical parameters. However, other parameters may be computed and other algorithms used.

Healthy male volunteers were studied. The probe consisted of a 30 cm long and 10 mm diameter plastic tube attached to the end of a dedicated bag. The cylindrical bag was 14 cm long and made of 50 μm thick polyurethane material. The bag could be inflated to a maximal diameter of 90 mm (CSA 6350 mm²) and corresponding volume of 900 ml without stretching the wall of the bag. The size of the bag was chosen on the basis of impedance planimetry studies where the CSA never exceeded 6000 mm² and MRI pilot studies where distension of the entire length of rectum by a 14 cm long bag was confirmed.

The probe contained one large channel for infusion and withdrawal of water. To secure complete emptying the bag this channel was connected to a special 10 cm long and 3 mm diameter flexible side holed tube positioned inside the bag. The infusion channel was connected to a 300 ml plastic syringe (as a safety precaution) that was filled from a 1000 ml container of sterilized, degassed and temperate water.

For bag pressure measurement the probe contained a 0.7 mm diameter channel ending inside the bag. The channel was continuously perfused (perfusion rate of 0.1 ml pr min) with degassed water by a low-compliance perfusion system. Using 6 m rigid non-compliant plastic tubing the pressure channel was attached to an external pressure transducer positioned outside the magnetic resonance (MR) scanner room. Recordings of pressure were amplified, analogue-to-digital converted and stored on a computer for later analysis.

The sensory intensity during bag distension was assessed using a 0-10 visual analogue scale (VAS) (0=no sensation, 5=pain threshold, 10=unbeatable pain). The volunteers were instructed how to use the scale and trained during the preconditioning distensions. The sensory intensity was assessed as the average of the VAS score obtained just before and after the each scan.

The subjects arrived at the MR scanner after overnights fast. Half an hour before the investigations they were given a sodium diphosphate enema to empty their rectum. After calibrating the equipment, the probe was passed into the rectum until a mark approximately 3 cm from the proximal edge of the balloon. The probe was fixated by tape in the same position during the entire study. The subjects were asked to lie in prone position inside the bore of a conventional MR scanner (Gyroscan Intera 1.5T, Philips, Best, the Netherlands) and to relax for 10 minutes. The pressure transducer was placed at the same level as the rectum. A minimum of four distensions was performed at volumes corresponding to 3-4 on the VAS to precondition the tissue and to adjust the MRI sequences. All distensions were performed without letting air into the bag system.

Stepwise distensions were then initiated by manually syringe infusion in 50 ml volume steps. The pressure was maintained for approximately two minutes during the image acquisition. The bag was emptied for minimum two minutes between each distension. The subjects scored the sensation intensity on the VAS. At maximum 5 on the VAS (pain threshold) the distensions were stopped.

Both sagittal scans and scans perpendicular to the centre axis of the rectum were obtained providing 30 images for each distension step with an image gap of 3.0 mm and pixel size of 0.39 mm.

Image Processing

The MR images were post-processed by customized software (Interactive Data Language 6.0, Research Systems Inc., Boulder, Colo., United States). The inner and outer contours of the rectal wall were identified for each cross-sectional image by semi-automatic edge detection based on greyscale threshold. Two experienced radiologists supported by altering slice directions and multi-planner reconstruction then adjusted and confirmed each contour manually. Consensus was obtained between the two radiologists.

Model reconstruction including solid model re-slicing and surface smoothing The inner and outer contours for each cross-sectional image were imported into and processed by MATLAB 6.5 software (The MathWorks Inc., Natick, Mass., United States). Hence, computation of the three-dimensional (3D) rectal surface was possible. This model was generated based on the transverse cross sectional images along the straight long axis of the stem part of rectum.

As indicated in the center axis of the organ was curved. Since the distended rectum was deformed along a curved axis the alignment of data points along a curved axis was necessary to describe the rectal deformation at different distension volumes. By dividing the 3D model into 30-39 equidistant segments along the curved center axis of the rectum the generation of a re-sliced solid 3-D model in any direction was possible. The reconstructed surfaces also had some irregularities due to the discretization of the images. The irregularities were reduced using a modified non-shrinking Gaussian smoothing method as outlined below.

Computation of Geometric and Biomechanical Parameters

The surface area and the volume for both the inner and outer contours were calculated based on the arc length and the cross sectional area as outlined below. The difference between the two volumes represents the volume of the rectal wall.

The circumferential strain was calculated based on the average of circumference in approximately the same 6 slices in both the stem and bending part of the rectum. The longitudinal strain was calculated in four different regions. In each region the longitudinal strain was based on the average length of approximately the same 10 longitudinal lines starting at the first and ending at the last slice. The strain ε was then calculated as the stretch ratio with the empty bag as reference length, ε=l/lₒ.

The rectum has a complex 3D geometry. Since the surface is smooth and continuous, it was approximated locally by a biquadric surface patch. Hence, the principal curvatures, tension and stress were analyzed using a surface fitting method as outlined in Appendix A. The peak tension was calculated as the highest tension in the entire rectal wall structure.

Based on the inner surface area A and the inner volume V of the 3D models a constructed bag length l was calculated based the on assumption of cylindrical shape l=A²/(V×4π). For evaluation of the present method an estimated radius r and tension T=p×r based on the assumption of both cylindrical r=\sqrt{V/(π×l)} and spherical r=³\sqrt{3}V/4π shape were calculated.

Surface Smoothing

Irregularities were removed using a modified non-shrinking Gaussian smoothing method. The relation between the position of the vertices before and after N iteration can be expressed as
X^{N}≦((I−μK)(I−λK))^{N}X (A1)

where N was the number of iterations, λ and μ are two scale factors, I is the n_{V}×n_{V} identity matrix, K=I−W, W is the weight matrix and n_{V} is the number of the neighborhood of a vertex. In this study, λ=0.1 and μ=−0.101 to −0.103 were selected as the scale factors. The iteration number ranged from 100 to 300 according to the criterion that the relative error between the volume calculated from the smoothed model and the volume calculated from the model must be less than 10%.

Calculation of Geometric Characteristics

The approximate surface area and the volume were calculated from:

$$Sarea = {\sum }_{i = 1}^{n - 1} 0.25 * ( {arc}_{i} + {arc}_{i + 1} ) * ( {h}_{max} + {h}_{min} ) \\ Volume = {\sum }_{i = 1}^{n - 1} 0.25 * ( {area}_{i} + {area}_{i + 1} ) * ( {h}_{max} + {h}_{min} ) \qquad ( A2 )$$

where arcᵢ and areaᵢ is arc length and cross sectional area at a given cross section i, hₘₐₓ and hₘᵢₙ are the maximum and the minimum height between cross sections i and i+1 and n is the number of slices.

Principal Curvatures Computation

Since the surface is smooth and continuous, it can be approximated locally by a biquadric surface patch. In this study, the local surface patch used is a tensor product B-spline surface as given by:

$$P ( u , v ) = {\sum }_{i = 0}^{2} {\sum }_{j = 0}^{2} {d}_{ij} {N}_{i}^{2} ( u ) {N}_{j}^{2} ( v ) \qquad ( A3 )$$

Each surface element consisted of 9 vertexes, three sequential points in the circumferential direction and three matching points (i.e., points originating from the same meridian). Thus, equation 3 can be expressed as:

$$X ( u , v ) = {\frac{1}{4} [ \begin{matrix}1 & u & {u}^{2}\end{matrix} ] [ \begin{matrix}1 & 1 & 0 \\ - 2 & 2 & 0 \\ 1 & - 2 & 1\end{matrix} ] [ \begin{matrix}{X}_{00} & {X}_{01} & {X}_{02} \\ {X}_{10} & {X}_{11} & {X}_{12} \\ {X}_{20} & {X}_{21} & {X}_{22}\end{matrix} ] [ \begin{matrix}1 & 1 & 0 \\ - 2 & 2 & 0 \\ 1 & - 2 & 1\end{matrix} ]}^{T} [ \begin{matrix}1 \\ v \\ {v}^{2}\end{matrix} ] ( u , v ∈ [ 0 , 1 ] ) \qquad ( A 4 )$$

u,v are the coordinates in a local tangent plane coordinate system. The X matrix is the coordinates of the nine vertexes.

Then, the principle curvatures and principle directions for the central point can be calculated from the coefficient of the first fundamental form (E, F and G) and the second fundamental form (L, M and N) of the differential geometry as:

| E = xᵤ² | L = −xᵤNᵤ | |
|---|---|---|
| F = xᵤxᵥ | M = −xᵤNᵥ | |
| G = xᵥ² | N = −xᵥNᵥ | (A5) |

where

$$N = \frac{{x}_{u} \times {x}_{v}}{\vert {x}_{u} \times {x}_{v} \vert}$$

is the normal vector to the surface and the subscripts indicate partial differential (for example, xᵤ is the partial differential of x with respect to u). The principal curvatures k₁ and k₂ can be combined from the Gaussian curvature (K_{G}) and the Mean curvature (K_{M}):

$$\begin{matrix}{K}_{G} = {k}_{1} {k}_{2} = \frac{L N - {M}^{2}}{EG - {F}^{2}} & ( A 6 ) \\ {K}_{M} = \frac{1}{2} ( {k}_{1} + {k}_{2} ) = \frac{1}{2} \frac{NE - 2 MF + LG}{EG - {F}^{2}} & ( A7 )\end{matrix}$$

K_{G} is a particularly useful curvature parameter that indicates an elliptical surface (K_{G}>0), a parabolic surface (K_{G}=0) or a hyperbolic surface (K_{G}<0) K_{M} is in inverse proportion to the surface tension according to the Laplace's Law p=T*(k₁+k₂) where p denotes the transmural pressure acting on the surface, T is the surface tension which was assumed constant in every direction and k₁ and k₂ are the principal curvatures. The stress at a given surface point was calculated according to S=T/h_{wall}, where S is the stress, T is the tension and h_{wall} is the wall thickness at the point.

Example of an Apparatus Comprising Means for Thermal Control of the Balloon

A thermal stimulation is created by circulating tempered water in the balloon of a probe. Depending on the temperature of the circulating water the stimulation vary.

FIG. 12 depicts a diagram of a thermal stimulation system using a peristaltic pump. The arrows indicate the flow of the water. In the box of metal (A) the temperature is controlled by the surrounding water. The filling tube (C) is the connection between A and the probe, likewise is the emptying channel (D). The flow in C and D is generated by a peristaltic pump (E1) which is forcing the tempered water from A to the probe and back. The flow in D is reversed compared to C, as shown in FIG. 12 which results in a circulation of water in the balloon of the probe.

As the diameter of C and D is different a pressure is build up. To locate the pressure in A the tube with the smallest diameter (highest resistance) must be connected as the filling channel (C).

If the circulation is started with leveled pressure a giving time will pass until the difference in pressure is stable. To avoid this time gab a third tube (D) is connected to A from where a syringe can create the pressure for steady state by drawing out a given amount of fluid. The amount is established empirically and varies depending on the difference in diameter between C and D. Since the peristaltic pump in off state closes C and D the pressure is created in A and not leveled in the system.

Example of a way to compute heat transfer coefficient for a bag placed in an organ Flux and coefficients due to temperature differences between the organ and the bag can be computed in different ways. In the following is an example of computing such parameters for evaluation of tissue properties and perfusion. According to the first law of thermodynamics, the total energy increase of the balloon system equals the heat received plus the work received:

$$\frac{ⅆ E}{ⅆ t} = \frac{ⅆ Q}{ⅆ t} + \frac{ⅆ W}{ⅆ t} \qquad ( 1 )$$

Where E is the system energy [J]
t is the time [s]
Q is the heat [J]
W is the work [J]

The work is assumed negligible so the convection heat loss equals the decrease of the energy.

$$\frac{ⅆ E}{ⅆ t} = \frac{ⅆ Q}{ⅆ t} \qquad ( 2 )$$

2. The energy decrease of the system is:

$$\frac{ⅆ E}{ⅆ t} = - m {c}_{p} \frac{ⅆ T}{ⅆ t} \qquad ( 3 )$$

Where m is the water mass inside the balloon [kg], cp is the water specific heat at constant pressure [J/kg·K] and

T is the water temperature at time t [K]

3. The convective heat loss can be defined as follows according to Newton's law of cooling:

$$\frac{ⅆ Q}{ⅆ t} = \overline{h} {A}_{w} ( T - {T}_{b} ) \qquad ( 4 )$$

Where h̅ is the average value of the heat transfer coefficient [W/m2K]

Aw is the balloon contact surface area [m2]

Tb is the body temperature assumed to be constant about 37° C. [K]

4. Therefore

$$\begin{matrix}\begin{matrix} \frac{ⅆ E}{ⅆ t} = \frac{ⅆ Q}{ⅆ t} \\ - m {c}_{ p} \frac{ⅆ T}{ⅆ t} = {h}^{ \_} {A}_{ w} ( T - {T}_{ b} )\end{matrix} & ( 5 ) \\ \frac{ⅆ T}{ T - {T}_{ b}} = - \frac{ {h}^{ \_} {A}_{ w}}{ m {c}_{ p}} ⅆ t & ( 6 )\end{matrix}$$

If h̅ is a constant, integration the above equation from time t=0 to t

$${\int }_{{T}_{0}}^{T} \frac{ⅆ T}{T - {T}_{b}} = {\int }_{0}^{t} \frac{\overline{h} {A}_{w}}{m {c}_{p}} ⅆ t \\ ln \frac{T - {T}_{b}}{{T}_{0} - {T}_{b}} = - \frac{\overline{h} {A}_{w}}{m {c}_{p}} t \\ T = {T}_{b} + ( {T}_{0} - {T}_{b} ) {ⅇ}^{- \frac{\overline{h} {A}_{w}}{m {c}_{p}} t} \qquad ( 7 )$$

Hence, the water temperature inside the balloon is an exponential decay function of time.

5. Curve fitting the T vs. time curve obtained from experiment to the exponential decay function thus, the constant value

$$\begin{matrix}a = \frac{\overline{h} {A}_{w}}{m {c}_{p}} or & ( 8 ) \\ ln \frac{{T}_{0} - {T}_{b}}{T - {T}_{b}} = at & ( 9 )\end{matrix}$$

can be obtained, where a is the slope of the line. Hence, the average heat transfer coefficient is defined as

$$\overline{h} = \frac{{amc}_{p}}{{A}_{w}} \qquad ( 10 )$$

If the balloon is assumed to be a cylinder of radius r [m] and length L [m], thus the heat transfer coefficient can be denoted as:
m=ρV=μπr²L
A_{w}=2π·L (11)

$$\overline{h} = \frac{{amc}_{p}}{{A}_{w}} = \frac{a \cdot ρ π {r}^{2} L \cdot {c}_{p}}{2 π r L} = \frac{a ρ r {c}_{p}}{2} \qquad ( 12 )$$

Where ρ is the water density [kg/m³]

V is the volume of the balloon [m³]

FIG. 13 shows an example of temperature experiments in the human esophagus where the temperature initially is 60 degrees Celsius and where it drops as function of time at volumes 10, 15 and 20 ml inside the bag.

Then the a value in Eq.(8) and (9) is 0.01172 and 0.01014 for volume 15 and 20. Thus the heat transfer coefficient for these two volumes can be obtained from Eq.12 as:

$${\overline{h}}_{v 1} = \frac{{a}_{v 1} ρ {r}_{v 1} {c}_{p}}{2}$$

$${\overline{h}}_{v 2} = \frac{{a}_{v 2} ρ {r}_{v 2} {c}_{p}}{2}$$

Example of a Complete Unit

An embodiment of the invention may be implemented as a unit that does the stimulations, pump infusions, data acquisition, data storage and handling. The unit can be connected to disposable multipurpose probes for measurement of parameters such as pressure along the organ, pH, luminal cross-sectional area, volume, etc and for stimulating the wall by distending a balloon or bag, perhaps even with the possibility of recirculating fluid in the bag for thermal stimulation, electrical stimulation, and with injection channels for chemical or pharmacological stimulations. The probes may be sold with all necessary utensils such as fluid to fill the balloon and perfuse pressure channels if required, chemicals and other items. Ideally the unit itself or a computer connected to the unit will provide the user with useful information about the organ such as the pressure pattern, whether the organ is dilated, stiff, hyperreactive, hypersensitive, etc. The user will use this information for learning more about the organ behaviour and for diagnostic purposes. It can ideally provide a quick indication of whether the patient has a dilated organ such as due to obstruction, cancer, achalasia, systemic sclerosis, or a hyperactive organ such as due to spasms of the muscles, or a hypersensitive organ such as may occur in non-cardiac chest pain. In a preferred embodiment the unit will contain 4-5 pressure channels/transducers, impedance measurement system for impedance planimetric measurement of cross-sectional area, fluid flow, contractility, axial deformation of the catheter (by providing measurement inside a fluid-filled channel of the catheter), or simply for detection of air bubbles or impeded flow in a channel of the probe. It may also contain pH sensors and chemical sensor at the surface of or close to the bag/balloon. The software for computation provides means for baseline adjustment, displaying simple tracings and for more advanced computation of tension, strain, active-passive tension strain curves, starling plots, velocity-tension plots, power plots etc. For example the contraction velocity may be obtained from evaluating the change in cross-sectional area (or rather by the change in circumference) per time unit during a contraction and relating this to the preload tension immediately before the contraction. The power plot is a multiplication of velocity and tension as function of the preload tension. All of these parameters may be combined with sensory data where the sensory data can be displayed as function of the parameters and relations can be described mathematically. The system will be highly automated and use predefined or userdefined protocols. Examples of such protocols are ramp distensions until the tissue is preconditioned. The degree of distension will be guided by the sensory data as obtained using a visual analogue scale or by other means. A pump in the unit will automatically reverse as soon as a predefined VAS level, volume or other end parameter is reached. The distension may then be followed by other stimulations or for the esophagus by swallow induced contractions. Ideally the user will buy one unit and boxes with a specified number of probes and utensils. The preferred probe is disposable which can be secured by an electronic or software mechanism. The box may only work for a certain number of studies before it has to be replaced also. The test should be so easy in terms of connecting the system, automatic precalibration, inserting the catheter to the correct position, starting the infusions, displaying the data in relation to normal values, and even providing a suggestion for diagnosis. Thus, the GP or medical specialists in private practice may use the system rather that referring the patients to the hospital laboratories. The equipment may contain various safety devices so the patient can himself disconnect the probe.

A typical test protocol in the esophagus can be like

Insert catheter

wait for 10 minutes

start recording of parameters such as pressures and sensory data

make 5 induced swallows and record pressure and pH

precondition the tissue mechanically by balloon distension

do 1-2 ramp distension test

infuse acid proximal to the balloon for 10 minutes

repeat the distension test

provide electrical stimulation from electrodes placed on the outside of the balloon take the probe out

The test can be done in various parts of the organ, even in sphincters. The data may be displayed and can be exported to other programs. Based on known normal values the unit will provide information of use such as the organ is dilated and with weakened peristalsis, acid reflux and hypersensitivity to the acid. This may be a guide for treatment of the symptoms.

Although the present invention has been described in connection with preferred embodiments, it is not intended to be limited to the specific form set forth herein. Rather, the scope of the present invention is limited only by the accompanying claims.

In this section, certain specific details of the disclosed embodiment such as material choices, geometry of the apparatus or parts of the apparatus, techniques, measurement set-ups, etc., are set forth for purposes of explanation rather than limitation, so as to provide a clear and thorough understanding of the present invention. However, it should be understood readily by those skilled in this art, that the present invention may be practised in other embodiments which do not conform exactly to the details set forth herein, without departing significantly from the spirit and scope of this disclosure. Further, in this context, and for the purposes of brevity and clarity, detailed descriptions of well-known apparatus, circuits and methodology have been omitted so as to avoid unnecessary detail and possible confusion.

1. A method of obtaining morphometric measures of a hollow internal organ, the method comprising the steps of:
introducing from an exteriorly accessible opening of a bodily hollow system a catheter into the hollow system, the catheter being provided with one or more inflatable balloons situated between a proximal end and a distal end of the catheter;
inflating at least one of the balloons in the hollow internal organ at least until the balloon abuts an inner wall of the hollow system, so as to obtain a level of inflation of the at least one of the balloons; and
determining at least one morphometric parameter at a level of inflation.

2-35. (canceled)

36. A method according to claim 1, wherein the determined morphometric parameter is selected from the group consisting of: wall thickness, layer thickness, inner and outer surface areas, areas of interfaces between layers and inner and outer circumferences, circumferences of interfaces between layers, wall volume, layer volume, wall cross-sectional area, layer cross-sectional area, luminal cross-sectional area, luminal diameter, and other measures.

37. A method according to claim 1, wherein the at least one morphometric parameter is obtained from medical imaging of the balloon.

38. A method according to claim 1, wherein the at least one morphometric parameter is obtained from medical imaging of the hollow organ.

39. A method according to claim 37 or 38, wherein the medical imaging is obtained by using an imaging technique selected from the group consisting of magnetic resonance scanning, X-rays and fluoroscopy in one or more planes, CT scanning, ultrasound, and other imaging means.

40. A method according to claim 1, wherein the balloon is inflated, while the organ is under the influence of relaxing or stimulating drugs or chemicals.

41. A method according to claim 1, wherein the morphometric parameter is correlated to a measurement of pressure in the balloon or dimension of the balloon, so as to obtain a correlation between the inflation level and the morphometric parameter.

42. A method according to claim 1, wherein the balloon is inflated, while a liquid or gas is infused into the organ under study.

43. A method according to claim 1, wherein at least one of the parameters: tensions, stresses, strains, or elastic stiffness, in one or more directions and possibly in different layers, is evaluated from equilibrium analysis or by finite element analysis of the morphometric parameter.

44. A method according to claim 1, wherein electrodes are placed on the outside of at least one of the balloons for establishment of at least one morphometric parameter by measurement of one or more wall impedances or conductances.

45. A method according to claim 44, wherein the one or more wall impedances or conductances are correlated to the level of inflation or derived from mechanical parameters.

46. A method according to claim 45, wherein the one or more wall impedances or conductances are correlated to the level of inflation or derived from mechanical parameters at resting conditions or during a natural bodily movement, or during physical stimulation with infused volumes, electrical stimulation, chemical stimulation, systemic or local infusion of drugs or other artificial stimulants, to provide information about the organ properties.

47. A method according to claim 1, wherein morphometric parameters are correlated with a feedback system comprising feedback selected from the group consisting of VAS data, electrical brain signals, electronic registration of referred pain data, longitudinal force measurements, and luminal pressures.

48. A method according to claim 1, further comprising analyzing the morphometric data to compute mechanical parameters using active-passive tension-strain analysis, active-passive stress-strain analysis, power plots, or preload-afterload muscle analysis, using algorithms in real time or offline with or without correlation to sensory data obtained by VAS scales or other means.

49. A method according to claim 1, wherein a temperature test is conducted and correlated to the level of inflation so as to establish morphometric parametersas a function of geometric and mechanical parameters selected from the group consisting of volume, cross-sectional area, diameter, length, pressure, force, tensions, stresses, and strains.

50. A method according to claim 49, wherein heat or cold flux through a tissue of the organ is obtained from temperature data obtained in the temperature test together with a determination of the contact area between the balloon and the tissue.

51. A method according to claim 49, wherein the heat or cold flux obtained at various levels of inflation is used to determine a tissue perfusion.

52. A method according to claim 49, wherein the temperature of the fluid in the balloon is controllably changed, preferably in a step fashion with or without changing the volume inside the balloon, and measured by temperature sensors placed inside or on the surface of the balloon to analyze the temperature change and time to reach a baseline as indicative of a tissue perfusion.

53. An apparatus for measurement of morphometric data of a bodily hollow system, the apparatus comprising a catheter comprising an inflatable balloon situated between a proximal end and a distal end of the catheter, the proximal end being in flow communication with the balloon, the balloon being configured to receive an inflating fluid from the proximal end and thereby inflate to a first and a second level of inflation, the first and second levels of inflation being determined by measurements of cross-sectional areas, pressures or volume of the balloon.

54. An apparatus according to claim 53, wherein the first and second level of inflation of the balloon is obtained from a shape of the balloon, the shape being obtained in an imaging of the balloon, the imaging is obtained by using an imaging techniques selected from the group consisting of magnetic resonance scanning, X-rays and fluoroscopy in one or more planes, CT scanning, ultrasound, and other means for imaging.

55. An apparatus according to claim 53, wherein the catheter comprises means for transmitting data to a data processing unit, and wherein the data transmission between the catheter and the data processing unit or between an intermediate unit and the data processing unit is wireless or alternatively is wired.

56. An apparatus according to claim 53, wherein the catheter is provided with an identifier so that a unique match to the data processing unit may be obtained.

57. An apparatus according to claim 53, wherein the data processing unit comprises a calibrator configured to match a catheter provided with an identifier.

58. An apparatus according to claim 53, further comprising means for infusion of cold or warm liquid or gas into the balloon in a step or ramp fashion and means for concomitant measurement of temperature, cross-sectional areas, pressures or volume.

59. An apparatus according to claim 58, further comprising means for measurement of sensory responses at various levels of inflation.

60. An apparatus according to claim 53, wherein the level of inflation is quantified by cross-sectional area measurements from at least one electrode placed inside the balloon, on the probe, or on the inside of the balloon, or by intraluminal or externally placed ultrasound or MR coils.

61. An apparatus according to claim 60, wherein said at least one electrode is selected from the group consisting of a conducting wire threaded through the catheter, a ring or partial ring of electrically conducting material, and a flexible circuit or silk print disposed along the catheter and at least partially wrapped around the catheter.

62. An apparatus according to claim 53, wherein the length of the balloon or bag can be changed by closing off part of the balloon or bag with a string or a smart device.

63. An apparatus according to claim 53, wherein the inflating fluid is recirculated in the balloon to control temperature in the balloon.

64. An apparatus according to claim 63, wherein the balloon is in flow communication with a thermal stimulator comprising a container submerged in a surrounding fluid and wherein the filling and emptying of the balloon is obtained by tubes with flow created by a pump.

65. A system comprising two or more apparatuses according to claim 53, wherein at least two different functionalities of at least one of the apparatuses can be selected by a user.

66. A system according to claim 65, wherein at least one of the apparatuses is provided in a sealed package comprising fluid to fill the balloon and perfuse pressure channels and chemicals.

67. A system according to claim 65, wherein the system is adapted to detect sensory data.

68. A method comprising:
introducing a catheter having a proximal end and a distal end into a hollow internal organ;
providing a balloon between the proximal end and the distal end of the catheter, the balloon being adapted to receive an inflating fluid from the proximal end of the catheter; and
stimulating a hollow internal organ by inflating the balloon to a first and second level of inflation.

69. A method comprising:
introducing a catheter having a proximal end and a distal end into a hollow internal organ;
providing a balloon between the proximal end and the distal end of the catheter, the balloon being adapted to receive an inflating fluid from the proximal end of the catheter;
inflating the balloon to a first and second level of inflation; and
measuring at least one morphometric parameter of at least part of the organ.
